(19) United States
(12) Patent Application Publication
(10) Pub. No.: US 2005/0124968 A1
(43) Pub. Date: Jun. 9, 2005

(54) Precision medication delivery method

(76) Inventor: Jon Davis Mollhagen, Lorraine, KS (US)

Correspondence Address: Bradley P. Sylvester;Suite 300, 200 North Broadway, Wichita, KS, 67202, US

(21) Appl. No.: 10/727,019

(22) Filed: Dec. 3, 2003

(51) Int. Cl. A61M 31/00

(52) U.S. Cl. 604/506

(57) ABSTRACT

A method for determining specific dosage levels for individual animals, in which the mass of an animal is determined, and where the dosage is determined in relation to the mass of the animal. The dosage information is used to determine the amount of medicinal substance that is to be pumped directly to an injector means or a spray means, where it can be applied or injected into the animal.

### BACKGROUND OF THE INVENTION

The administration of medications to livestock is a requirement in modem cattle operations, especially those involving feedlots which are prime areas for the spread of unchecked diseases. It is important to not only administer medications, but to administer them at the proper time and at the proper dosage. Previous methods have concentrated on the determination of proper medications using data, and assuming that the medications themselves will be dispensed as desired. Human error is a factor that data cannot account for, and simply using animal weight and determining what type of medication is necessary is insufficient, where the medication is closely tied in terms of dosage level to the target animal's mass. The measurement of dosage levels is optimally not approximated or manually obtained.

Referring to U.S. Pat. No. 6,606,966 ( Teachey et al.), a precision medication dispenser is disclosed, which involves the administration and dispensing of medication using a hopper and a counter. This patent examples the lack of automation, that removes human error.

Referring to U.S. Pat. No. 5,273,528 (Skeen et al.), an applicator for dispensing medicinal fluids is shown. It is a manual apparatus, and requires direct administrative interaction for each use. This injector systems used with livestock lacks the automation necessary to remove human error, and reduce human effort in filling the applicator.

The mixture of medications with water is also exemplified in U.S. Pat. No. 4,248,176 (Kilstofte), in which the medical materials are metered into a holding tank, exemplifying the need for distribution of medications, but where the actual medication dosage is not controlled for individual animals.

Referring also to U.S. patent application No. US 2003/0188689 (Pratt), an example of cattle management methods and systems are shown. This type of management system deals with the computation and optimal measurements of food and the other products to maximize the weight gain of livestock. This method requires that a particular animal be identified and distinguished from every other animal in a group. Specific feed measurements and projected target weight gain are set, and the method deals with working toward the target weight. This method, which follows other prior applications by the same inventor, does not deal with the actual delivery of specific medications in a manner that the present invention does. The automated techniques in the present invention remain beyond the scope of this patent, or other similar patents.

Referring also to U.S. Pat. No. 6,592,517 (Pratt et al.), a method and system for providing animal health histories and tracking inventory of related drug usage is shown. This particular method is directed toward the “assisting an operator in selecting and deciding whether to administer a selected drug treatment . . . ”. Data entry is required, and updating animal health history is also a basis in this patent. The methodology used for the actual introduction of medication is not covered, other than comprising the use of data to assess the overall animal situation.

Referring now also to U.S. Pat. No. 6,516,746 (Pratt et al.), this patent is a continuation of other previously filed patents of similar nature. The method described and claimed concerns a comprehensive system for maximizing weight gain for a target animal, and involves directing the animal to a specific feeding location. Identification of the animal's mass is one of the steps noted in the method, but this determination is used to direct procedures to further increase the animals mass, and not to determine the proper dosage of medication during this method process.

Other patents exist, with regard to the methods and systems for providing animal health histories and tracking inventory of medications. In U.S. Pat. No. 5,315,505 (Pratt et al.), identifying a particular animal and determining of the dosage for various medications comprise a portion of the method steps. This method, involves treating the dosage levels as data to be considered and entered into an overall tracking system. The method described here does not automatically cause certain amounts of medications to be dispensed with appropriate apparatus. It is assumed that the medications will be dispensed as the data indicates, and this still leaves room for human error.

### SUMMARY OF THE INVENTION

It is important to the proper maintenance and care of livestock that proper inoculations and medications be dispensed. The methods of inoculation administration typically include the selection of each individual animal and either guessing as to the proper dose, or simply administering a set dose to each animal.

Medication effectiveness is often dependent on proper dosage as related to the mass of the animal. There is generally an optimal dosage level described per units mass for each type of medication. Certain medications lose effectiveness if an insufficient dosage is given. Likewise, dosage that is excessive may provide no more effectiveness than a proper dosage level.

Since most medication dispensing techniques and methods generally require that person administering the medication to guess or estimate the mass of the animal, it is typical for animal mass estimates to be higher, rather than lower. This often results in wasted medications due to over inoculation.

Medication levels for particular animals are often distributed and administered through a manually set injector gun. Where dosage is related to mass, the mass of the target animal is determined as accurately as possible. Typically, the animals will have a mass of the few hundred pounds to 700 or more pounds. Dosages are often metered in increments related to every 100 pounds of mass. Such rounding of mass levels with regard to dosage amounts does not provide optimal use of medications, especially where the medications are expensive.

It is a further aspect of importance that medication levels be accurately provided, and that the estimation and delivery of the medication be as accurate as possible. Removing the guesswork and estimation factors involved, and using data about the animal along with mechanical dispensing apparatus, dosage level variations outside of the desired parameters is avoided.

The typical inoculation process involves placing the target animal into a squeeze chute, so that the animal cannot move around in an undesired manner. Animal identification procedures are implemented, if necessary. Various animals may be identified through transponders, tags, or other indicia. Identification of the animal is not required, and this information may be utilized where necessary to promote the proper records of immunization and medication dispensing.

The target animal mass is determined, using a load sensor or other mass determining apparatus. The animal's mass is determined and sent to a control system. The control system will determine the proper dosage for the animal, and will activate an appropriate pump to either fill a spray reservoir, or an injection apparatus.

In either situation, a precision dispensing pump meters the dosage to the application apparatus, and the medicine is either injected through the activated injection gun, or sprayed in the appropriate manner. The person doing the application and dispensing of the medications does not control the dosage level, unless they override the automated system. This allows not only an accurate means to deliver the precise optimal amount of medication, but removes the factor human of human error. Human error comprises misreading data, inadvertently measuring incorrect amounts of medical substances, as well as removing the tendency of a person who is failing to attend to their duties appropriately from making any errors whatsoever when it comes to applying and/or delivering medicine.

### DESCRIPTION OF THE FIGURES

FIG. 1 depicts a flow chart of the method.

FIG. 2 depicts the apparatus used with an injection means with the method.

FIG. 3 depicts the spray portion of the apparatus used with the method.

### DETAILED DESCRIPTION OF THE INVENTION

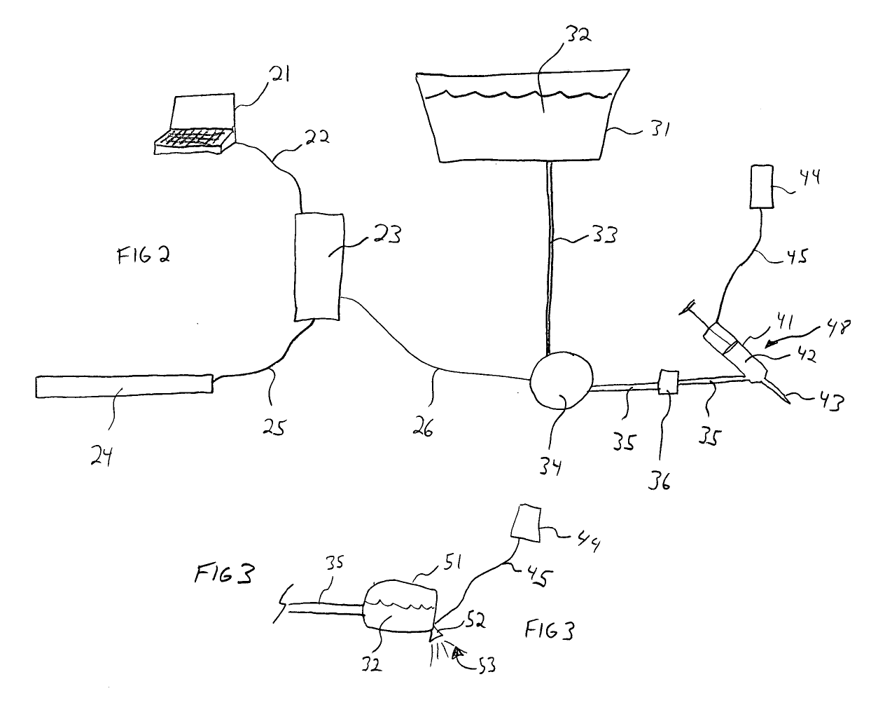

An improved method for dispensing medicinal substances is shown and described below. Referring to FIG. 2, an apparatus system is shown, using the method described below. A data entry terminal 21 allows data about the type of medication being used to be entered into the memory of the master controller 23, being communicated through any means commonly known and understood in the art, and shown as one method by being transmitted through electrically conductive wire 22. The terminal 21 may be a separate data entry point from the master controller 23, or it may be incorporated directly into the master controller 23, so as to be an attached portion of it.

The master controller 23 uses the data from the terminal 21, and utilizes programming which computes the appropriate amount of medical substance to be administered, in proportion to the weight or mass of the animal. This computation is made by receiving the weight or mass of the animal from scale 24, transmitted to master controller 23, which is then entered into a preset program that assesses the previously determined optimal medication amount for that particular weight. The animal so weighed is weighed individually, and the medically optimal dosage is determined for that specific animal. The animals may be any type of livestock, or animal raised in a group setting, such as cattle, chickens, horses, hogs, or other similar animal groups.

The master controller 23, after determining the optimal dosage, transfers the dosage amount to pump 34, using any means commonly known and understood in the art. The method shown in FIG. 2 comprises a wire 26, but is intended to convey a path of travel for the data.

Pump 34 receives the dosage amount data. This data may comprise input information that the pump uses to determine how much medical substance to pump, or may be comprised of electrical signals that strictly turn the pump 34 on and off. In either instance, the pump 34 directs medical fluid 32 stored in a reservoir 31, and which is able to be received by pump 34, through inlet tubing 33, into pump outlet tubing 35 to the applicator gun 48. The applicator gun 48 comprises a chamber 42 enclosed within the applicator body 41, where the applicator 48 is able to vary the volume of the chamber 42. Any medical substance 32 in the applicator is intended to comprise a single dose for a single animal. The applicator gun 48 is directed to the animal, and the dosage received from the pump 34 is injected into the animal. The applicator 48 is either able to be moved manually, or through a mechanical actuating system 44, that is operated by the person monitoring the applicator 48, and who causes the actuating of the applicator gun 48 to propel the medical substance 32 through the needle 43 into the animal.

Referring now also to FIG. 3, a spray applicator 53 is shown, which may be used in place of the applicator gun 48. Using the procedures noted above for the applicator gun 48, the medicinal substance 32 is pumped through outlet tubing 35 and into the pump reservoir 51, where it is ejected through spray nozzle 52. The spraying process is actuated through the actuating system 44 that transmits a signal through transmitting means 45, to have the sprayer 53 eject the contents of the medicinal substance 32 in the reservoir 51, where said contents of medicinal substance 32 were metered using the procedures and methods described above.

Once the target animal has received the medicinal substance 32, the animal is released, and a new animal may take its place.

The metering of the medicinal substance 32 may be controlled through a valve system 36, which is placed as an intersection in the line 35, and which controls the amount of medicinal substance to be allowed to enter into the applicator 48 or sprayer 51.

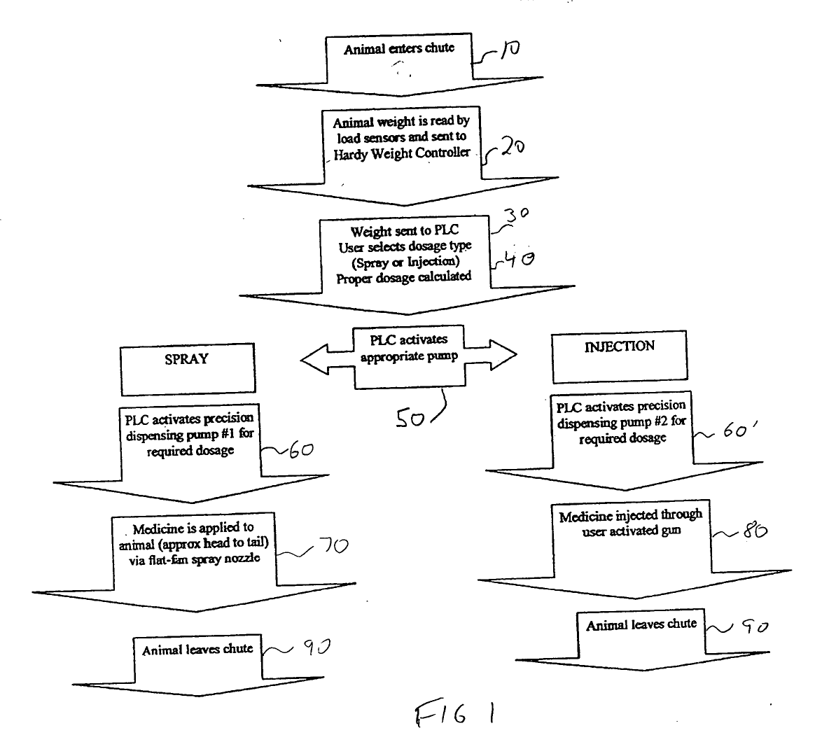

Referring also to FIG. 1, this method of metering specific amounts of medicinal substances comprises the following steps.

First, a target animal is selected, and separated from other animals 10. This can be done in a variety of methods. The typical method would comprise a situation where the target animal enters a specific chute. With other animals, such as chickens, or other domestic animals, suitable holding pens and apparatus are commonly known in the art that allow the animal to be immobilized for purposes of mass/weight determination, and for the receiving of medications. The primary objection in the selection is to direct a specific animal into an area where it can be immobilized, such as a chute for a cow or steer.

Once the animal is suitably immobilized, it can be adequately weighed 20. Typically, an animal such as a cow will be put into a squeeze chute, so that the animal is unable to shake our move around, thus disrupting mass determination. Load sensors are typically used, as is commonly known in prior art, to determine the animal's mass.

Once the animal's mass is determined, that information is sent to a PLC, also known as a controller, as input data 30.

This data is entered with regard to the specific animal, if necessary, and shows the history of the animal with regard to its previous management and previous medications.

Using previous history where necessary, and also using the mass determined for the animal, a proper dosage of the specific medication needed is determined 40.

Once the specific dosage is determined, the PLC controller automatically activates the appropriate pump 50, which will pump 60 the appropriate amount of medication, using dispensing pump #1 into the injector. If a sprayer system is to be used, the PLC will activate the precision pump to dispense the appropriate amount of medication to a system sprayer.

Some systems may have both a spray apparatus and an injection apparatus, with injection systems having one or more actual injectors. Where multiple injectors are available, the controller will cause the appropriate amount of desired medication to be sent to the appropriate injector.

The medication is applied to the animal, either through spray 70, generally being applied head to tail to the animal, or through injection 80 using a user activated gun. In this manner, the dosage applied is not dependent on the accuracy of a human individual determining anything about the animal, other than it is positioned to receive the medication. This removes the need for human involvement to fill syringes accurately, as well as to accurately determine the optimal dosage level.

Once the target animal has received the medication, it is allowed to leave the chute area 90.

From the foregoing statements, summary and description in accordance with the present invention, it is understood that the same are not limited thereto, but are susceptible to various changes and modifications as known to those skilled in the art and we therefore do not wish to be limited to the details shown and described herein, but intend to cover all such changes and modifications which would be encompassed by the scope of the appended claims.

1. A method for administering specific amounts of medicinal substances to animals, using an injection apparatus having steps comprising:
a. placing an animal into an area where is can be immobilized for purposes of obtaining an accurate determination as to mass;
b. weighing the animal to determine mass and sending this data to a controller;
c. computing the correct dosage of medicinal substances relating directly to the actual weight of the animal within the controller, and using this data to determine the metering requirements for the medicinal substances;
d. activating a pump that will dispense a metered amount of medicinal substance as the determined dosage level to an injector means;
e. inserting the injection means into the animal and injecting the medicinal substance into the animal;

2. A method for administering specific amounts of medicinal substances to animals, using an injection apparatus, as recited in claim 1, having the additional step of selecting a specific animal to be given a dosage of medicinal substances prior to placing the animal within the area where it can be immobilized.

3. A method for administering specific amounts of medicinal substances to animals, using an injection apparatus, as recited in claim 1, in which a separate metering means is used to restrict the amount of medicinal substance to the injector means.

4. A method for administering specific amounts of medicinal substances to animals, using an injection apparatus, as recited in claim 1, in which the injector means is actuated manually.

5. A method for administering specific amounts of medicinal substances to animals, using an injection apparatus, as recited in claim 1, in which the injector means is actuated mechanically.

6. A method for administering specific amounts of medicinal substances to animals, using an injection apparatus, as recited in claim 1, in which the medicinal substance is stored in a reservoir, and where the contents of said reservoir are able to be received by the pump.

7. A method for administering specific amounts of medicinal substances to animals, using an injection apparatus, as recited in claim 1, in which the data for determining proper dosage as to weight is input into the controller prior to weighing the animal.

8. A method for administering specific amounts of medicinal substances to animals, using a spray apparatus having steps comprising:
a. placing an animal into an area where is can be immobilized for purposes of obtaining an accurate determination as to mass;
b. weighing the animal to determine mass and sending this data to a controller;
c. computing the correct dosage of medicinal substances relating directly to the actual weight of the animal within the controller, and using this data to determine the metering requirements for the medicinal substances;
d. activating a pump that will dispense a metered amount of medicinal substance as the determined dosage level to a sprayer means;
e. positioning the sprayer means so that the medicinal substance can be applied onto the animal;

9. A method for administering specific amounts of medicinal substances to animals, using a spray apparatus, as recited in claim 8, having the additional step of selecting a specific animal to be given a dosage of medicinal substances prior to placing the animal within the area where it can be immobilized.

10. A method for administering specific amounts of medicinal substances to animals, using a spray apparatus, as recited in claim 8, in which a separate metering means is used to restrict the amount of medicinal substance to the injector means.

11. A method for administering specific amounts of medicinal substances to animals, using a spray apparatus, as recited in claim 8, in which the sprayer apparatus is actuated manually.

12. A method for administering specific amounts of medicinal substances to animals, using a spray apparatus, as recited in claim 8, in which the sprayer apparatus is actuated mechanically.

13. A method for administering specific amounts of medicinal substances to animals, using a spray apparatus, as recited in claim 8, in which the medicinal substance is stored in a reservoir, and where the contents of said reservoir are able to be received by the pump.

14. A method for administering specific amounts of medicinal substances to animals, using a spray apparatus, as recited in claim 8, in which the data for determining proper dosage as to weight is input into the controller prior to weighing the animal.
